(19) United States
(12) Patent Application Publication
(10) Pub. No.: US 2009/0054628 A1
(43) Pub. Date: Feb. 26, 2009

(54) METHOD AND SYSTEM FOR IN VITRO PROTEIN FOLDING

(76) Inventors: Richard St. John, San Francisco, CA (US); Jeffrey Luk, Emeryville, CA (US); Thucdoan Le, San Leandro, CA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/260,965

(22) Filed: Oct. 29, 2008

Related U.S. Application Data
(63) Continuation of application No. 11/495,142, filed on Jul. 28, 2006.
(60) Provisional application No. 60/703,647, filed on Jul. 29, 2005.

(51) Int. Cl. C07K 14/565; C07K 1/00; B01J 19/00

(52) U.S. Cl. 530/351; 530/402; 422/224

(57) ABSTRACT

A method of recovering a refolded protein involves static mixing a concentrated solution of a denatured protein with a refolding diluent to obtain the refolded protein. The method is particularly suitable for microbially produced recombinant proteins in large processing volumes. The denatured protein solution can be obtained by isolating protein from the microbial host and exposing them to a denaturant. This solution is mixed with a suitable refolding diluent under static mixing conditions compatible with proper folding of the protein so that the refolded protein is obtained, preferably rapidly and with high yield. A system for implementing the refolded protein recovery method includes a static mixer, a conduit inline with and upstream from the static mixer, and an inlet to the conduit upstream of the static mixer, and optionally a dynamic, preferably non-turbulent, mixing vessel downstream from the static mixer. The invention finds particular use in large scale production of proteins, particularly recombinant proteins.

### CROSS-REFERENCE TO RELATED APPLICATIONS

This application claims priority to U.S. Provisional Patent Application No. 60/703,647, filed Jul. 29, 2005, titled METHOD AND SYSTEM FOR IN VITRO PROTEIN FOLDING, the disclosure of which is incorporated herein by reference in its entirety and for all purposes.

### BACKGROUND

1. Technical Field

This invention is in the general area of protein chemistry. More specifically it relates to methods and systems for refolding a protein produced by recombinant technology.

2. Related Art

Typical commercial production schemes for recombinant proteins involve the transformation of a cell, often a bacterial cell such as Eschericia coli (E. coli), to produce a foreign product, often of mammalian origin. The gene that encodes the protein is inserted into the host cell and is translated into the corresponding protein through normal cell mediated production. A bacterial host cell, however, may be unable to correctly fold such a recombinant protein since it lacks the environment and organelles present in a mammalian cell to do so. As a result, cells may produce aggregates of unfolded or improperly folded proteins. When produced in high concentrations, the unfolded and partially folded proteins may begin to form insoluble aggregates or agglomerated, insoluble entities known as inclusion bodies. These amass in the periplasmic space and can, at times, make up more than 50% of the bacterial cell's total protein. Much of the inclusion body is made up of the protein of interest (sometimes yielding over 90% purified protein), making it already highly purified, with small molecules, host cell proteins, and nucleic acids making up the remainder of the inclusion body.

The advantages of producing recombinant protein in an E. coli cell rather than a mammalian cell, given the misfolding that often occurs, are that bacterial cells are readily available, grow much faster and can overproduce the protein of interest. They are also unable to harbor certain viruses that can be found in mammalian cells. Work, then, has gone into attempting to purify and properly refold the protein from E. coli thereby making the product economical and safer for human injection.

After isolating the protein, for example from aggregates or inclusion bodies, the first step in purifying the protein is to solubilize it in a strong salt concentration, for example 6M guanidine-hydrochloride (GuHCL) or 8M urea. Both salts are chaotropic reagents that dissolve and unfold the protein by breaking hydrogen bonding and hydrophobic interactions holding the inclusion body together. See e.g., Ladisch, Michael R, Bioseparations Engineering: Principles, Practice and Economics (2001) John Wiley and Sons, Inc., 118-123. In addition, a reducing reagent, such as dithiothreitol, cysteine or beta-mecaptoethanol may be needed to break disulfide bonds incorrectly linked during production of the protein. The unfolded protein solution is subsequently diluted or dialyzed with a refolding buffer (possibly containing oxido-shuffling reagents to assist in disulfide bond formation) to reduce the denaturant concentration, allowing the protein to refold using its innate chemical structure.

A major pathway of product loss during the refolding step is aggregation. Aggregation occurs when the attractive forces between separate proteins are more favorable than the attractive forces between protein and solute. Subsequently, the favorable intramolecular residue-to-residue attractions, which help refold the protein to its native state, compete with the unfavorable intermolecular attractive forces, resulting in soluble aggregates. These soluble aggregates may then accumulate and lead to the precipitation of insoluble aggregates. While aggregation at times can be a reversible reaction, attempting to refold aggregates is undesirable as it increases production times and costs. Hence, once formerly aggregated or agglomerated proteins are solubilized, further aggregation is generally to be avoided.

To date, the detailed mechanisms of protein refolding and aggregation are complex and continue to be debated. It is known that refolding does not occur in one step; instead, the protein follows discrete conformational changes as the denaturant is removed. At these intermediate conformations between the unfolded and folded state, pathways of refolding, aggregation or misfolding (another pathway for product loss) compete. Environmental conditions and the innate chemical structure of the protein help to dictate which competing pathway will dominate during refolding.

Attempts at avoiding aggregation during refolding have been made by changing the environmental conditions, including protein concentration, denaturant concentration and localized temperature, of a protein-solute mixture. For example, the kinetics of the aggregation reaction have been found to be of a higher order than the refolding reaction with respect to protein concentration (Kieffiaber, T., Rudolph, R., Kohler, H.-H., Buchner, J. “Protein Aggregation in vivo: A Quantitative Model of the Kinetic Competition Between Folding and Aggregation.” Bio/Technology, 1991, 9, 825-829). For this reason, refolds are often performed under dilute conditions relative to the solubility limit. Under such conditions, the chance for the molecules to come into contact with each other and the possibility of attraction is reduced.

Experimentally, proteins also exhibit a tendency to aggregate during refolding at intermediate denaturant concentrations. When at an intermediate conformation, the protein could have exposed areas that have a potential to aggregate at its hydrophobic residues, as described above. Refolding then could fail if the denaturant is removed or decreased too slowly.

Additionally, thermal stress to the protein will increase the likelihood of protein aggregation during refolding. It appears that the aggregation reaction is suppressed for many proteins at low temperatures while for other proteins, that reportedly refold at higher temperatures, aggregation may not be a significant pathway. The mechanism of this refolding/aggregation behavior has not yet been conclusively established. It may be due to a temperature dependence of hydrophobic forces involving shielding of the nonpolar surfaces between proteins (see, e.g., Baldwin, R. L. Temperature dependance of the hydrophobic interaction in protein folding. Proc. Natl. Acad. Sci., 1986, 83, 8069-8072) or another separate pathway for intermediates that are aggregation prone.

From what is known about aggregation during refolding, the concentrated material requires rapid mixing with the diluent buffer to avoid any localized areas of high protein concentration, denaturant concentration and temperature. Current experimental procedures involve the use of a pitched blade impellor in an unbaffled tank to rapidly combine a concentrated form of solubilized protein with a diluent buffer. This type of dynamic mixer is the most commonly used device in industry for vigorous mixing. The mixer is initially set to stir at turbulent speeds to induce a vortex in the diluent buffer. A dropper is then aimed at the vortex or directly at the impeller, which slowly delivers the concentrated protein solution.

Scaling up the mechanical mixer, though, has proved to be challenging. Studies have shown that at low agitation rates where the mixing is not turbulent, regions of isolated mixing form (Makino, T., Ohmura, H., Kataoka, K. Observation of Isolated Mixing Regions in a Stirred Vessel. Journal of Chemical Engineering of Japan, 2001, 34 (5), 574-578). In these regions, the protein is too highly concentrated and in danger of aggregation, as described above. The solution then is to stir at highly turbulent speeds to avoid regions of isolated mixing during refolding. However, given the high amount of shear stress from the impeller due to subsonic pulses and localized cavitation near the trailing edges of the blade, the protein may experience higher mechanical denaturing stress as the process is scaled up. See e.g., Fennema, Oreg. 1996. Food Chemistry. 3rd Edition. Marcel Dekker, Inc., New York. Chapter 6. Furthermore, high agitation rates create a high power input into the system thereby producing a possible thermal denaturing stress to the protein via power dissipation.

Improved protein refolding processes, including improved mixing schemes, for large scale production of proteins, particularly recombinant proteins, would be desirable.

### SUMMARY OF THE INVENTION

The present invention addresses these needs by providing a method of refolding protein by statically mixing a concentrated solution of a denatured protein with a refolding diluent to obtain a mixture with the refolded protein. The method is particularly suitable for microbially produced recombinant proteins in large scale processing volumes, such as 30 L or more, for example up to 200 or 1000 or even 10,000 L. The denatured protein solution can be obtained by isolating protein from the microbial host and exposing them in a denaturant. This solution is mixed with a suitable refolding diluent under static mixing conditions compatible with proper folding of the protein so that the refolded protein having biological activity is obtained rapidly and with high yield. The invention finds particular use in large scale production of proteins, particularly recombinant proteins.

The invention also provides a system suitable for implementing the protein refolding method of the present invention. The system includes a static mixer, a conduit inline with and upstream from the static mixer, an inlet to the conduit upstream of the static mixer, and a low shear dynamic mixing vessel downstream from the static mixer. In operation, a source of refolding diluent is delivered to the conduit upstream of the static mixer, and a source of concentrated denatured protein is delivered to the conduit via the inlet upstream of the static mixer. Following static mixing, the solution is retained in the low shear dynamic mixing vessel for a period of time to optimize process yield. The static mixer includes a series of mixing elements in a conduit. The mixing elements may be fixed or moveable, but are un-powered (i.e., static) and provide mixing action only by the movement of the liquid flow over them.

These and other aspects and features of the invention will become more fully apparent when the following detailed description of the invention is read in conjunction with the accompanying drawings.

### BRIEF DESCRIPTION OF THE DRAWINGS

FIG. 1 is a simplified schematic diagram illustrating the main features of a static mixer for use in accordance with the present invention.

FIG. 2 is a block diagram illustrating the main features of a system for recovering a refolded protein from a solution of the denatured protein in accordance with the present invention.

FIG. 3 is a flow diagram illustrating a method for recovering a refolded protein from a solution of the denatured protein in accordance with the present invention.

FIG. 4A is a representative plot of the concentration of denaturant versus the fraction of unfolded protein in solution.

FIG. 4B is a representative plot of time versus the fraction of mixed protein (mixing behavior and rate) for dynamic and static mixing.

FIG. 5 is a plot of time vs. % activity illustrating the result of the experiment described in Example 2, below.

### DESCRIPTION OF SPECIFIC EMBODIMENTS OF THE INVENTION

The methods and systems of the present invention will now be described with reference to several embodiments. Important properties and characteristics of the described embodiments are illustrated in the structures in the text. While the invention will be described in conjunction with these embodiments, it should be understood that the invention it is not intended to be limited to these embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail in order not to unnecessarily obscure the present invention.

Introduction

The present invention provides a method of recovering a refolded protein by statically mixing a concentrated solution of a denatured protein with a refolding diluent to obtain a mixture with the refolded protein. The method is particularly suitable for microbially produced recombinant proteins, such as Interferon β-1b, in large processing volumes, such as 10 L, 30 L or 100 L or more, for example up to 200 L or 1000 L or even 10,000 L. The denatured protein solution can be obtained by isolating protein from the microbial host and exposing them to a denaturant. This denatured protein solution is mixed with a suitable refolding diluent under static mixing conditions compatible with proper folding of the protein so that refolded protein having biological activity is obtained rapidly and with high yield. The invention finds particular use in large scale production of refolded proteins, particularly recombinant proteins.

The invention also provides a system for implementing the refolded protein recovery method of the present invention. The system includes a static mixer, a conduit inline with and upstream from the static mixer, and an inlet to the conduit upstream of the static mixer. In operation, a source of refolding diluent is delivered to the conduit upstream of the static mixer, and a source of concentrated denatured protein is delivered to the conduit via the inlet upstream of the static mixer. The static mixer includes a series of mixing elements in a conduit. The mixing elements may be fixed or moveable, but are un-powered (i.e., static) and provide mixing action only by the movement of the liquid flow over them.

Static Mixing

Protein mixing is a coupling of two phenomena: reduction of localized areas of possible aggregation prone environments, and increase of mechanical stress on the protein. Thus when the mixing is low, shear on the protein is low but the protein may experience localized high protein concentrations allowing it to aggregate. When the mixing is high, aggregation from the environment is less likely, but the mechanical shear on the protein is high, possibly damaging the protein. There is an optimum level of mechanical mixing that needs to be determined to find a midpoint between shear and localized areas of aggregation prone environments during refolding.

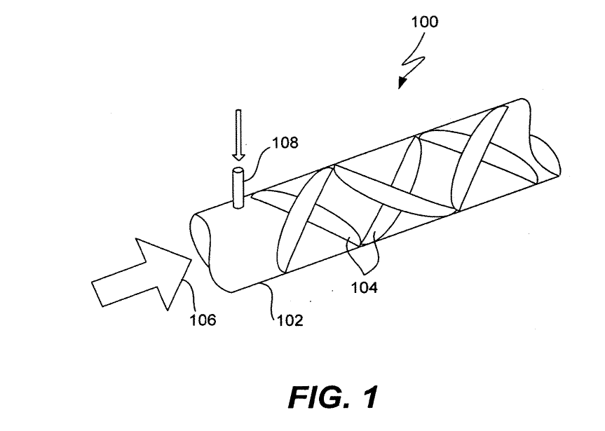

The present invention provides an innovative approach to the challenges of effective mixing for protein folding with static mixing. A static mixer is a series of geometric elements within a conduit (e.g., a pipe) configured to create mixing between two or more fluids flowing through the mixer using the energy of the fluid flow. Static mixing, then, is the mixing of two or more flowing fluids using only the energy of the fluid flow. The geometric mixing elements can be any conformation of materials fitted inside the static mixer conduit which result in mixing of a fluid stream passed over the elements. The elements are often fixed, but they can move as long as any movement of the elements is as a result of the movement of the fluid to be mixed over the elements rather than an external power source. Preferred examples include blades and helices. Referring to FIG. 1, a typical static mixer 100 is a combination of a pipe 102 and a set of fixed helical elements 104 that separates a fluid stream and then mixes the resulting streams through a gentle vortex. This series of events continue at each element. Fluids to be mixed are supplied to the mixer 100 via a major inlet 106 in line with the mixer, typically for the larger volume of the fluids, and a lesser inlet 108 entering through the wall of the conduit just upstream from the mixing elements 104. Mixing occurs gently but rapidly, avoiding any localization of salt concentration, temperature and protein concentration.

A suitable static mixer for the system of the present invention may have a variety of geometries and configurations that may, at least in part, be dependent on processing volumes. For volumes in the 30-200 L range, conduit having a diameter of from less than ¼ inch up to 2 inches have been found to be acceptable, for example 3/16 inch, ¾ inch, 1 inch and 2 inches. A suitable static mixer may have between about 2 and 20 mixing elements, for example between 6 and 12 mixing elements, for example 6 or 12 elements. The elements may be fixed or moveable or a combination. The elements may have any suitable shape and configuration. In specific embodiments the static mixer has 6 or 12 fixed helical elements. Such static mixers are available from Koflo Corporation, Cary, Ill., for example.

Such a mixer is suitably operated as follows for a protein refolding application: The initial concentrated protein can be at as low a concentration as desired, but it is generally at a concentration of about 10 mg/ml of denaturant and may be higher, for example up to about 20 mg/ml or more as long as solubility is maintained. The denaturant may be about 3-10 M, such as 5 M or 8 M, Gu-HCL or urea, for example. The concentrated, denatured protein solution is diluted in the static mixer to the point where refolding occurs. 10, 20, 30 or 60 fold dilutions may be conducted, for example.

In general, the refolding diluent is a buffer in which the protein is soluble and that promotes proper folding of the protein. This may be protein specific and, while suitable refolding buffers are known for many proteins, some degree of experimentation, well within the expertise of those skilled in the art, may be required to determine the appropriate buffer to act as a refolding diluent for a given protein. Some examples of buffers suitable as refolding diluents in accordance with the invention include about 5 mM glycine with pH about 3, about 5 mM phosphoric acid with pH about 2-3 and about 2 mM aspartic acid with pH about 4.

A suitable temperature range for the process is between about 2 and 30° C., for example about 2-8° C., such as 4° C. Where not inconsistent with protein stability, room temperature is preferable so that refrigeration apparatus is not required.

The flow rate of the mixture is chosen such that the static mixer has a Reynolds Number of between about 200 to 7000. The process is capable of achieving a yield of greater than 75% monomer (or at least 80, 85, 90, 95, 97, 98 or 99%) in less than one day, or less than one hour, for example less than 30 minutes, or less than 5 minutes. While not limiting the invention, it is believed that percentage monomer is correlated with the ability of the protein to carry out a biological function associated with the mitigation of a medical disorder, referred to herein as biological activity or biologically active.

Using a static mixer in protein refolding can reduce aggregation during protein folding by two mechanisms:

Static mixers quickly and efficiently blend fluid streams to rapidly dilute concentrated denatured protein into the refolding diluent. For example, a suitable static mixer can generally mix 30-200 L of fluid in less than 30 minutes, for example about 20-30 minutes; this is relative to the approximately 6 hours or more required for dynamic mixing of these large volumes. Consequently, transient concentrations of denaturant that support highly aggregation prone species (e.g., molten globules) are greatly reduced with the use of a static mixer. The threshold agglomeration concentration will vary by protein. For example, for Interferon β, pockets of concentration above about 0.2 mg/ml, (for example 0.1 mg/ml is acceptable), should be avoided. For TFPI, concentrations above about 2 mg/ml, should be avoided. Generally, a concentration as high as possible without risking agglomeration problems is preferred to minimize processing volumes.

Also, the static mixer creates a lower stress environment for the protein than dynamic mixing. For a stirred tank, the period of time in which protein experiences high shear is proportional to scale, since the bulk refold solution is continuously being vortexed throughout the addition of protein to diluent. The protein could experience anywhere from a few minutes to hours of prolonged stress as the process is scaled up in a stirred tank. In a static mixer, however, the protein is rapidly blended with the diluent (typically within seconds), and then exits to a low shear mixing vessel creating a shorter residence time of high mixing and thus a lower stress.

Another important advantage of the static mixer is its efficiency in power needed to drive the process. As stated previously, a negative impact caused by a power increase is that a greater amount of heat due to power dissipation is added to the system. Energy requirements for an agitated tank and a static mixer were compared for a 200 L process. Typical energy requirements for a stirred tank would be approximately 2-5 HP/1000 gal (Rushton, J. H., Costich, E. W. and Everet, H. J. Power Characteristics of Mixing Impeller, Part 1, Chemical Engineering Progress, 1950, 46, 467), while the energy requirements for a static mixer are approximately 0.005 HP.

The corresponding change in temperature can then be calculated using Equation 3:

$$Power = \frac{{mC}_{p} Δ T}{t} \qquad Equation 3$$

where m is the mass, Cp is the specific heat, T is temperature and t is time. Given a 20 min processing time, assuming all power is converted to heat and that the liquid is isolated, we find:

ΔT_{agitated vessel}˜0.22° C. and

ΔT_{statin mixer}˜0° C.

While these potential heating effects seem very small when averaged over the tank, local heating affects arise near the impeller creating high temperatures, which could be a significant concern during processing.

To scale up the static mixer, it is helpful and often necessary to determine a numerical value for mixing. Mixing for fluids is reliant on a characteristic length scale of the mixing zone and a characteristic velocity of the mixing species. On a macromolecular scale in a pipe, the mixing zone can be defined as the diameter of the pipe and the velocity defined as the flow rate of the entering fluid. Thus if we keep flow rate and the diameter of the pipe proportional, we keep mixing constant upon scale up. A scaling factor commonly used to relate velocity and diameter is the Reynold's number. For a static mixer, this is given by Equation 1:

$$Re = \frac{3157 QS}{μ D} \qquad Equation 1$$

where Q is the flow rate (gal/min), S is the specific gravity, μ, is the viscosity (cP), and D is the inner diameter of the pipe.

System and Method

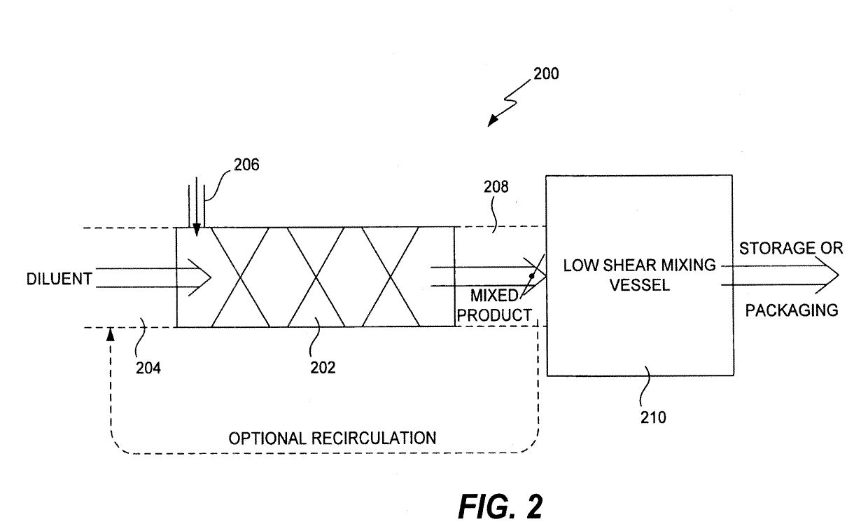

FIG. 2 is a block diagram illustrating the main features of a system for recovering a refolded protein from a solution of the denatured protein in accordance with the present invention. The system 200 includes a static mixer 202. The static mixer 202 is connected in line with a conduit (e.g. a pipe) 204, generally having about the same diameter as the static mixer. The conduit 204 provides an inlet to the static mixer for the larger volume of two fluids to be mixed in the static mixer, in this case the protein diluent. A second inlet 206 to the static mixer is provided for the smaller volume of the two fluids to be mixed in the static mixer, in this case the denatured protein solution.

A suitable static mixer for the system of the present invention may have a variety of geometries and configurations that may, at least in part, be dependent on processing volumes. For volumes in the 30-200 L range conduit has a diameter of from less than ¼ inch up to 2 inches have been found to be acceptable, for example 3/16 inch, ¾ inch, 1 inch and 2 inches. A suitable static mixer may have between about 2 and 20 mixing elements, for example between 6 and 12 mixing elements, for example 6 or 12 elements. The elements may be fixed or moveable or a combination. The elements may have any suitable shape and configuration. In specific embodiments the static mixer has 6 or 12 fixed helical elements.

The static mixer 202 outlets to a second conduit 208, typically a continuation of the first conduit 204. The second conduit 208 connects with a dynamic mixing vessel 210 so that the mixed protein product outlet by the static mixer can be conveyed to the dynamic mixing vessel 210 via the second conduit 208 to complete the folding process. Optionally, the statically mixed protein product may be re-circulated through the static mixer 202 via another conduit (pipe) one or more times prior to being routed to the dynamic mixing vessel. The dynamic mixing vessel is generally operated to avoid shear induced damage to the protein. For example, the dynamic mixing may be non-turbulent. Refolded protein in large volumes and high yield may then be collected from the dynamic mixing vessel 210 for storage or packaging as a pharmaceutical product. In this way, static mixing, achieves optimal protein mixing, and ultimately proper folding occurs rapidly without high concentration pockets, shearing or heating, and with low power consumption.

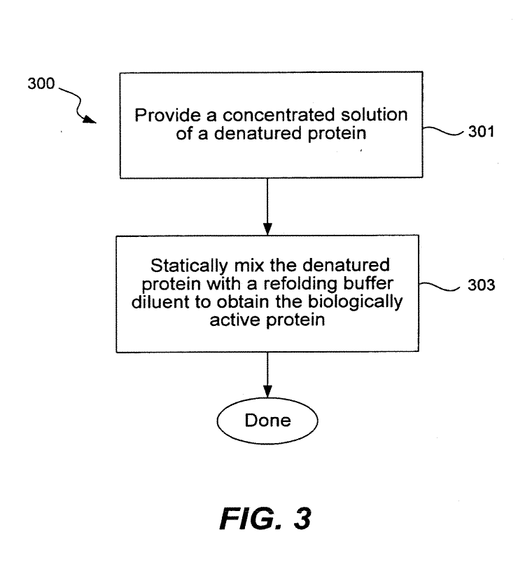

FIG. 3 is a flow diagram illustrating a method for recovering a refolded protein from a solution of the denatured protein in accordance with the present invention. The method involves providing a concentrated solution of a denatured protein (301) and statically mixing the denatured protein with a refolding diluent to obtain the refolded protein (303). As noted above with reference to the system of the invention, in a preferred embodiment the protein folding continues following the static mixing operation in a low shear mixing vessel.

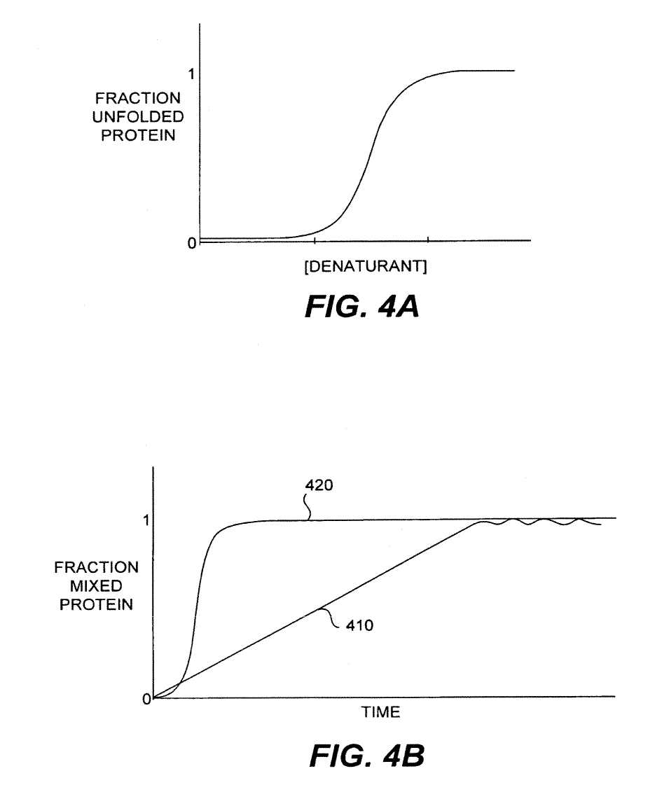

FIG. 4A is a representative plot of the concentration of denaturant versus the fraction of unfolded protein in solution. The plot illustrates that there is a relatively narrow range of denaturant (e.g., GuHCl) concentration over which a denatured protein folds. Dynamic mixing occurs gradually so that the folded condition of the protein in a solution being dynamically mixed follows the curve in the plot and there is imprecise control over the process mixture condition. Also, agglomeration problems are more likely to occur when the protein mixture is in a partially folded state, so reducing the amount of time the protein mixture spends in that state would be advantageous. Static mixing occurs much more rapidly, with the folded condition of a statically mixed protein solution moving along the curve effectively in a point-to-point (unfolded to folded) manner with very little time spent in the intermediate partially mixed state. This provides for a much greater degree of control, consistency, and therefore robustness to the process, allowing for the fine control of kinetics to achieve thermodynamics optimized for native protein folding.

FIG. 4B is a representative plot of time versus the fraction of mixed protein (mixing behavior and rate) for dynamic and static mixing. This plot further illustrates the point noted above with reference to FIG. 4A of the relative rates of mixing achieved by dynamic versus static mixing. Dynamic mixing, represented by curve 410, occurs only gradually, resulting in a lengthy state intermediate mixing, while static mixing, represented by curve 420, occurs rapidly.

Formulation

The method and system of the present invention may also be used to incorporate excipients into a protein mixture to produce a therapeutic formulation of the active protein. For example, in accordance with the present invention, trehalose may be added to the fluids to be mixed.

One particularly advantageous application of the present invention is in the large scale production of HA-free recombinant proteins, such as Interferon-β 1b. Albumin is believed to complex with IFN thereby preventing IFN-IFN agglomeration. Removal of albumin to create an HA-free IFN formulation exacerbates the agglomeration problem during mixing. While the invention is not limited by this theory, it is believed that trehalose may mitigate some of the agglomeration problems induced by removal of albumin in HA-free protein formulations. In one embodiment, a protein solution to be mixed includes 0.25 mg/ml HA-free Interferon β-1b in 2 mM aspartic acid buffer at a pH of about 4 and 9% trehalose. The result of the process is a complete HA-free protein (e.g., IFN-β 1b) formulation.

### EXAMPLES

The following examples are provided to illustrate certain aspects of the present invention. The examples will serve to further illustrate the invention but are not meant to limit the scope of the invention in any way.

### Example 1

### Renaturation of Protein with One Disulfide Bond

One protein of interest for commercial production is Interferon-β (IFN-β), in particular Interferon-β 1b (IFN-β 1b), a 18.5 kD synthetic, recombinant protein analog of IFN-β. IFN-β 1b is a refolded protein which has the cysteine residue at position 17 replaced by a serine residue. As a microbially produced protein, IFN-β 1b is unglycosylated. It also has an N-terminal methionine deletion. It is characterized by a very hydrophobic surface in the native state and by one disulfide bond, which remains intact throughout processing. IFN-β 1b, marketed as Betaseron®, has been formulated into a successful pharmaceutical that has been approved for treatment and management of multiple sclerosis (MS). This protein analog, materials and techniques for its manufacture, its formulation as a therapeutic and its use to treat MS are described and claimed in a number of US patents and applications including Application No. 435,154, filed Oct. 19, 1982; U.S. Pat. No. 4,588,585, issued May 13, 1986; U.S. Pat. No. 4,737,462, issued Apr. 12, 1988; and U.S. Pat. No. 4,959,314, issued Sep. 25, 1990; each of which is incorporated by reference herein in its entirety and for all purposes.

In addition, some IFN-β pharmaceutical formulations, including Betaseron®, contain human serum albumin (HA or HSA), a common protein stabilizer. HA is a human blood product and is in increasingly low supply. Accordingly, more recently there has been a desire for HA-free drug formulations.

This experiment examined the feasibility of using a static mixer for refolding HA-free IFN and tested it over a variety of variables (including differing Reynolds numbers, tee distances and temperatures) for their effect on percent monomer, that is percent of properly folded protein with no intermolecular bonds. Percent monomer was determined by size exclusion chromatography HPLC. The variables tested were compared to percentage of monomer obtained with a mechanical mixing process under similar conditions.

To refold IFN at the 0.1 g scale, a 10 mg/mL HA-free IFN-β 1b solution containing SM guanidine hydrochloride (GuHCl) was diluted 60× with a diluent in a 2-8° C. cold room. This experiment initially used the Koflo 12-element 3/16″ disposable inline mixer which was modified to include a reducing hose barb adapter (tee) approximately 1 mm upstream of the mixing elements. Later trials were performed using the Koflo 6-element ⅗″ inline mixer described below for the 1 g scale. Reynold's numbers tested were between values of 300 and 2000. A control was run with 8 mL IFN solution added to 472 mL refolding diluent vigorously mixing on a stir plate. Results at this stage are found in Table 1, below.

TABLE I
Results for IFN at 0.1 g Scale

| | Process | Mixer | Tee | | |
|---|---|---|---|---|---|
| | Time | Type | Distance | Temperature | % Monomer |
| RE = 1815 | ~3 min | 3/16″ | <1 cm | Cold Room | 98.35 |
| | | Plastic | | (2-8° C.) | |
| | | w/12- | | | |
| | | element | | | |
| RE = 331 | ~16 min | 3/16″ | <1 cm | Cold Room | 99.27 |
| | | Plastic | | (2-8° C.) | |
| | | w/12- | | | |
| | | element | | | |
| RE = 867 | ~6 min | 3/16″ | <1 cm | Cold Room | 98.27 |
| | | Plastic | | (2-8° C.) | |
| | | w/12- | | | |
| | | element | | | |
| Dynamic | ~30 min | N/A | N/A | Cold Room | 97.44 |
| Mixing | | | | (2-8° C.) | |

At the 1 g scale, 10 mg/mL IFN was diluted 60× with refolding diluent in a 2-8° C. cold room. Initial experiments used a Koflo 6-element3/5 disposable inline (static) mixer with a reducing tee connected between 2.5″ to 4″ before the inlet. Later trials used the ¾″ stainless steel static mixer described below at the 5 g scale. Reynold's numbers tested were between 2000-7000. The flow rates were stopped after a floor scale read the final desired volume. Results at this stage are found in Table II, below.

TABLE II
Results for IFN at 1 g Scale

| | Mixer Type | Tee Distance | Temperature | % Monomer |
|---|---|---|---|---|
| RE = 7000 | ⅗″ Plastic w/ | 2.5″ | Cold Room | 99.22 |
| | 6-elements | | (2-8° C.) | |
| RE = 2000 | ⅗″ Plastic w/ | 2.5″ | Cold Room | 97.30 |
| | 6-elements | | (2-8° C.) | |
| RE = 2000 | ⅗″ Plastic w/ | 4″ | Cold Room | 98.74 |
| | 6-elements | | (2-8° C.) | |
| RE = 2000 | ¾″ Plastic w/ | 3″ | Cold Room | 98.60 |
| | 6-elements | | (2-8° C.) | |
| RE = 5000 | ¾″ Plastic w/ | 3″ | Cold Room | 98.77 |
| | 6-elements | | (2-8° C.) | |

At a 5 g scale, 10 mg/mL concentrated IFN was diluted 60× with refolding diluent. A ¾″ KoFlo 6-element stainless steel static mixer was fined with a ½″ reducing tee just upstream of the mixing elements. The mixer was clamped to a bottom port of a SOL jacketed stainless steel tank with a wall impeller running on the opposite side. Using the average total flow rate, the Reynold's number was approximately 4000 for all static mixer runs. The concentrated IFN pump was stopped after entire contents were emptied from the concentrated IFN bottle while the buffer pump (for the refolding diluent) was stopped after a floor scale read the final desired volume (which included the holdup volume between the buffer tank and the refolding tank). Processing of material was at 2-10° C. After processing, the refold tank was left to mix for not less than 10 minutes. An additional experiment was performed using a 2″ Koflo stainless steel 6-element static mixer fitted with a ¾″ reducing tee. The Reynold's number was kept constant at 4000. Results are shown in Table III, below.

TABLE III
Results for IFN at 5 g Scale

| | Process | | Tee | Temper- | % |
|---|---|---|---|---|---|
| | Time | Mixer Type | Distance | ature | Monomer |
| RE = 4000 | ~10 min | ¾″ Stainless | 3″ | ~10° C. | 97.81 |
| | | Steel w/6- | | | |
| | | elements | | | |
| RE = 4000 | ~10 min | ¾″ Stainless | 3″ | ~5° C. | 98.36 |
| | | Steel w/6- | | | |
| | | elements | | | |
| RE = 4000 | ~3 min | 2″ AL6XN | 3″ | ~5° C. | 99.50 |
| | | w/12- | | | |
| | | element | | | |
| Dynamic | ~30 min | N/A | N/A | ~4° C. | 98.02 |
| Mixing | | | | | |

As shown in Tables I and III, final percent monomer was greater than control percentages for each scale. No control was run at the 1 g scale; however, each trial produced similar or greater yields than either control percentages for the 0.1 g scale and 5 g scale. Thus, the static mixing-based process and system of the present invention achieves at least as good, and usually better, yield as a conventional process. Another benefit is that at larger scales (i.e., 30 grams scale for manufacturing) static mixing time remains constant (3-15 minutes) whereas dynamic mixing time increases (from 30 minutes to several hours) due to concerns of localized protein or denaturant concentrations where aggregation of IFN could occur. Thus, static mixing is highly scalable to large-scale production. It also provides more consistent results and is a more robust process.

### Example 2

### Renaturation of Protein with Three or more Disulfide Bonds in Native State

Hen Egg-White Lysozyme is a 14% molecule with four disulfide bonds. It has a complex refolding scheme but is well studied and has been characterized in detail. This experiment shows that the static mixer will work for more complex folding schemes than IFN.

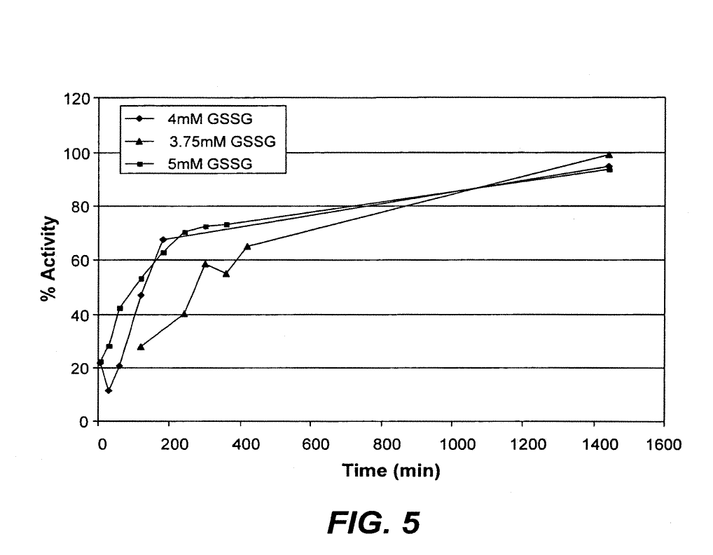

0.3 g lysozyme denatured in 8M GuHCl—/, 50 mM Tris, 1 mM EDTA, 32 mM DTT buffer, pH 8 at 37° C. for 1 hr was diluted 16-fold in 5 minutes with 1.25M GuHCl, 50 mM Tris, 1 mM EDTA buffer, pH 8 and incubated for 24 hr at 25° C. to yield a final concentration of 1 mg/mL. This experiment used a 3/16″ disposable static mixer with 12 helical elements that was fitted with a reducing hose barb adapter just upstream of the mixing elements. Flow rates were chosen to give a Reynold's number of approximately 1000 (i.e., 60 mL/min for diluent buffer and 4 mL/min for denatured lysozyme solution). Samples were taken for percent purity by activity assay to assess refolding kinetics. FIG. 5 is a plot of time vs. % activity illustrating the refolding kinetics for three separate refolds.

A final analysis of lysozyme kinetics was performed after 24 hours using a procedure adapted from Jolles, P. Lysozymes from Rabbit Spleen and Dog Spleen. Methods in Enzymology 1962, 5, 137. Final recovery of active lysozyme after 24 hours was 90% or greater for three separate trials. This is comparable to published results in which dynamic mixing yielded approximately 95% lysozyme activity (De Bernardez-Clark, E., Hevehan, D., Szela, S., Maachupalli, J. Oxidative Renaturation of Hen Egg-White Lysozyme. Folding vs Aggregation. Biotechnology Progress 1998, 14, 47-54). This experiment, thus, shows utility in refolding recombinant proteins with multiple disulfide bonds using a static mixer.

Discussion of Experimental Results

A major problem in refolding protein from inclusion bodies is aggregation. Aggregation can be described by attractive forces resulting in aggregation competing with attractive forces resulting in refolding. To control aggregation during refolding, vigorous mixing is employed. However, common mixing schemes using a mechanical mixer may either damage the protein or inefficiently mix the protein causing aggregation. The use of a static mixer is an innovative solution to this problem as it rapidly mixes streams without the extreme shear caused by mechanical mixing and can easily be employed in a manufacturing facility as it is easily scaled. Results from two separate proteins suggest a wide range of applicability.

### CONCLUSION

The static mixing-based process and system of the present invention achieves at least as good yield as conventional processes. In addition, it is highly scalable to large scale production, faster, provides more consistent results and is a more robust process.

Although the foregoing invention has been described in some detail for purposes of clarity of understanding, it will be apparent that certain changes and modifications may be practiced within the scope of the appended claims. It should be noted that there are many alternative ways of implementing both the processes and compositions of the present invention. Accordingly, the present embodiments are to be considered as illustrative and not restrictive, and the invention is not to be limited to the details given herein, but may be modified within the scope and equivalents of the appended claims.

All documents cited herein are hereby incorporated by reference herein in their entirety and for all purposes.

What is claimed:

1. A method of refolding a protein, comprising:
providing a concentrated solution of a denatured protein; and
statically mixing the denatured protein with a refolding diluent to obtain a mixture comprising refolded protein.

2. The method of claim 1, wherein the protein is a microbially produced recombinant protein.

3. The method of claim 2, wherein the denatured protein solution is obtained by isolating protein from the microbial host and exposing it to a denaturant.

4. The method of claim 1, wherein the refolding diluent is a buffer in which the protein is soluble.

5. The method of claim 4, wherein the static mixing occurs in a static mixer comprising a series of mixing elements in a conduit.

6. The method of claim 5, wherein the protein solution is delivered to a flow of the refolding diluent in a conduit immediately prior to the flow reaching the mixing elements of the static mixer.

7. The method of claim 1, wherein the mixture is provided to a dynamic mixing vessel following the static mixing.

8. The method of claim 7, wherein the mixture is dynamically mixed in the dynamic mixing vessel.

9. The method of claim 1, wherein the refolded protein is obtained with a yield of greater than 75% monomer in less than 1 hour.

10. The method of claim 9, wherein the refolded protein is obtained with a yield of greater than 95%.

11. The method of claim 10, wherein the refolded protein is obtained in less than 30 minutes.

12. The method of claim 11, wherein the refolded protein is obtained in less than 5 minutes.

13. The method of claim 4, wherein the concentration of the denaturant in the protein solution is at least 3M.

14. The method of claim 1, wherein the dilution is at least 10 fold.

15. The method of claim 14, wherein the dilution is about 60 fold.

16. The method of claim 1, wherein the temperature during mixing is between about 2 and 30° C.

17. The method of claim 1, wherein the temperature during mixing is about 4° C.

18. The method of claim 1, wherein the protein concentration during mixing is less than 0.2 mg/ml.

19. The method of claim 18, wherein the protein concentration during mixing is about 0.1 mg/ml.

20. The method of claim 1, wherein the flow rate of the mixture is chosen such that the static mixer has a Reynolds Number of between about 200 to 7000.

21. The method of claim 1, wherein the volume of the mixture is greater than 10 L.

22. The method of claim 1, wherein the volume of the mixture is greater than 100 L.

23. The method of claim 1, wherein the volume of the mixture is greater than 100 L.

24. The method of claim 1, wherein protein has one or more intra-molecular disulfide bonds in its native form.

25. The method of claim 24, wherein protein has one intra-molecular disulfide bond in its native form.

26. The method of claim 25, wherein protein is an Interferon β.

27. The method of claim 26, wherein protein is an Interferon β-1b.

28. The method of claim 27, wherein protein solution comprises an HA-free Interferon β.

29. The method of claim 1, wherein refolded protein is biologically active.

30. The method of claim 1, wherein the mixture further comprises excipients generally recognized as safe ingredients of a therapeutic formulation of the active protein.

31. The method of claim 30, wherein the excipients comprise trehalose.

32. The method of claim 31, wherein the protein solution comprises 0.1 mg/ml HA-free Interferon β-1b in 2 mM aspartic acid at a pH of about 4 and the excipients comprise 9% trehalose.

33. A system for recovering a refolded protein from a solution, comprising:
a static mixer;
a conduit inline with and upstream of the static mixer; and
an inlet to the conduit upstream of the static mixer.

34. The system of claim 33, further comprising:
a source of refolding diluent configured for delivery to the conduit upstream of the static mixer; and
a source of concentrated denatured protein configured for delivery to the conduit via the inlet upstream of the static mixer.

35. The system of claim 34, wherein the static mixer comprises a series of fixed mixing elements, moveable mixing elements, or a combination thereof in a conduit.

36. The system of claim 35, wherein the static mixer conduit has a diameter no more than 2 inches.

37. The system of claim 36, wherein the static mixer conduit has a diameter of about ¼ inch.

38. The system of claim 35, wherein the static mixer has fixed elements.

39. The system of claim 33, further comprising a dynamic mixing vessel downstream of the static mixer.
